SEQUENCE LISTING (6 sequences)

SEQ ID NO: 1 - DNA, 4675 bp, adeno-associated virus 2
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgtacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctccggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccggccatggcaagccacaaggacgatgaagaaaagttttttcctcagagcggggttctcatctttgggaagcaaggctcagagaaaacaaatgtgaacattgaaaaggtcatgattacagacgaagaggaaatcggaacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacacggtcagcgtggagatcgagtgggagctgcagaaggaaaacagcaaacgctggaatcccgaaattcagtacacttccaactacaacaagtctgttaatcgtggacttaccgtggatactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 2 - DNA, 145 bp, adeno-associated virus 2
  misc_structure (1)..(145): ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 3 - DNA, 47 bp, adeno-associated virus 2
  misc_feature (13): Unpaired base
cggcccgctggtttccagcgggctgcgggcccgaaacgggcccgccg

SEQ ID NO: 4 - DNA, 21 bp, adeno-associated virus 2
  misc_feature (10)..(12): Unpaired bases
cgggcgacctttggtcggccg

SEQ ID NO: 5 - DNA, 21 bp, adeno-associated virus 2
  misc_feature (10)..(12): Unpaired bases
cgcccgggcaaagcccgggcg

SEQ ID NO: 6 - DNA, 5 bp, Artificial Sequence
  misc_feature (1)..(5): Cyclic DNA
gcttt

(19) United States
(12) Patent Application Publication
(10) Pub. No.: US 2003/0100115 A1
(43) Pub. Date: May 29, 2003

(54) Cytotoxic agents

(76) Inventors: Kenneth Raj, Fribourg (CH); Peter Martin Beard, Epalinges (CH)

Correspondence Address: NIXON & VANDERHYE, PC, 1100 N GLEBE ROAD, 8TH FLOOR, ARLINGTON, VA, 22201-4714, US

(21) Appl. No.: 10/240,198

(22) PCT Filed: Apr. 20, 2001
(86) PCT No.: PCT/GB01/01795

(30) Foreign Application Priority Data
Apr. 20, 2000 (GB) 0009887.1

(51) Int. Cl. A61K 48/00; C12N 15/861

(52) U.S. Cl. 435/455; 514/44; 435/456

(57) ABSTRACT

A method of killing a cell that is lacking in effective p53 protein activity, particularly as compared to wild type, is provided characterised in that it comprises delivering to the cell a single stranded DNA including a portion with at least one base, internally located with respect to any 3′ and 5′ ends of the DNA, that is unbasepaired with another base in a form that is capable of being internalised by the cell.

The present invention relates to cytotoxic agents that have use against cells that lack p53 functionality (p53(−)), either wholly or partly, particularly being effective against p53(−) tumour cells and cells that have been infected by viruses that downregulate or eliminate the activity of p53 protein. The actions of these agents against cells infected with viruses make them effective anti-viral agents, particularly against viruses such Human Papilloma Virus (HPV).

A major goal of molecular oncology is to identify means to kill cells lacking p53 function. The p53 tumour suppresser gene encodes a nuclear phosphoprotein which is a multi-functional transcription factor involved in the control of cell cycle progression, DNA integrity and cell survival in cells exposed to DNA-damaging agents with resultant cancer-inhibiting properties. The development of human cancer often involves inactivation of p53 suppressor function through mechanisms including gene deletions and point mutations, which in turn lead to introduction of oncogenic mutations in other DNA. (See for example Greenblatt et al., 1994. Cancer Res., 54: 4855-78; Harris-CC, 1996. Carcinogenesis, 17: 1187-98; Ko-L and Prives-C, 1996. Genes Dev., 10: 1054-72; Levine-AJ, 1997. Cell, 88: 323-331).

The WHO body, the International Agency for Cancer (IARC/CIRC) 150 Cours Albert Thomas, F-69372 Lyon cedex 08, France, provides and maintains a database of over 8000 somatic p53 mutations in human tumours and cell lines. IARC reports that point mutations are scattered over more than 250 codons and are common in many forms of human cancer. As many as 90% of mutations reported in the IACR database are found in the core domain. Mutations at five “hotspot” codons (175, 245, 248, 249 and 273) represent about 20% of all mutations found so far.

An important activity of p53 is its ability to bind DNA. The p53 DNA-binding domain is made of two anti-parallel 13-sheets forming a “scaffold” supporting a DNA-binding surface of non-contiguous loops and helixes. Mutations can be grouped in three broad classes according to their impact on the structure of the DNA-binding domain. Class I mutations affect residues of the DNA-binding surface, such as Arg 248 and Arg 273, and disrupt protein-DNA contact points. Class II affect residues crucial for the correct orientation of the DNA-binding surface, such as Arg175 and Arg249, which are involved in the connections between the scaffold and the binding surface. These mutations may disrupt the regulation of p53 protein flexibility. Class III mutations fall within the “scaffold” and disrupt the tertiary structure of the whole DNA-binding domain.

It would appear that mutants corresponding to these categories have distinct functional properties as well as cell type-specific properties (Greenblatt-MS, Grollman-AP and Harris-CC, 1996. Cancer Res., 56:2130-36; Harris-CC, 1996. J Natl. Cancer Inst., 88: 1442-55; Ory et al., 1994, EMBO 13: 3496-3504 and Forrester et al., 1995, Oncogene, 10: 2103-2111). Differences in patterns of p53 mutations in several types of cancer reflect the effect of specific carcinogens (Greenblatt et al., 1994. Cancer Res. 54: 4855-78; Harris-CC, 1996. Carcinogenesis. 17: 1187-98). Well-characterized examples of such “mutagen fingerprints” include G:C to T:A transversions in lung cancers in association with cigarette smoke, G:C to T:A transversions at codon 249 on the third nucleotide in liver cancers in association with dietary exposure to aflatoxin B1 (AFBI) and CC:GG to TT:AA tandem dipyrimidine transitions in skin cancers in association with UVB exposure. The presence of p53 antibodies in the serum of some cancer patients may provide an interesting tool for diagnosis and follow-up of cancer (Soussi, 1996. Immunol. Today 17: 354-356).

Publications relating to DNA damage and the importance of p53 in cell fate decision include Bunz et al., 1998 Science (282): 1497-1501; Waldman et al., 1997 Nature Medicine (3)9: 1034-1036; -Suganuma et al., 1999 Cancer Research (59): 5887-5891 and Muschel et al., 1998 Oncogene (17): 3359-3363 (Review)

In making the present invention the present inventors have determined that adeno-associated virus (AAV) selectively kills cells that lack wild type, ie. intact, p53 activity. In her thesis of November 1999, the inventors coworker, P M Ogston, described experiments showing that U-2 OS osteoblasts with intact p53 and pRb activity (p53+), but pl6-, undergo arrest in the G2 phase of the cell cycle when infected with AAV, while Saos-2 osteoblasts lacking fully active p53 (p53−) and pRb (pRb−) arrest but then are killed. In U-2 OS, the arrest in G2 is characterised by an increase of p53 activity coupled with the targeted destruction of CDC25C—features that are identical to those induced by etoposide, a DNA-damaging agent Most surprisingly, she reported that AAV inactivated by ultraviolet irradiation, such that it can no longer produce proteins and replicate its DNA, exhibits enhanced ability to arrest both cells and enhanced ability to kill the Saos-2 cells, while viral-encoded proteins or viral particles without DNA, or adenovirus containing double-stranded DNA, was ineffective. It was concluded that something about Saos-2 rendered them vilunerable to AAV induced apoptosis and that this was likely caused by Rep protein associated with the viral DNA.

Adeno-associated virus (AAV) is a small, non-enveloped virus whose DNA of 4.7 kb is linear and single-stranded, with hairpin-like structures at both ends (1; FIG. 1a). AAV DNA encodes the three proteins, VP1-3, which make up the viral capsids and four non-structural proteins called Rep78, Rep68, Rep52 and Rep40, which control replication and transcription of the viral genome (2). Although Rep proteins are not required to assemble the viral particle, Rep is found associated with the particle (3). AAV is classified as a dependovirus because in order to replicate efficiently, it requires co-infection by another virus (e.g. adenovirus or herpes virus). To date, AAV has not been associated with any human disease, is relatively non-immunogenic and none of its proteins is known to possess oncogenic properties. Instead, it has been reported to suppress cell division (4-8). There have been reports that Rep78 may interact with p53 such as to suppress adenoviral oncogenic activity (Batchu et al. Cancer Res (1999) August 1; 59(15) 3592-5.

AAV DNA is single-stranded with hairpin structures at both ends (see FIG. 1 herein) that has been implicated in prophylaxis against tumours in the past. Of particular interest are papers by Schlehofer (1994) Mutation Research 305, 303-313 and by De la Maza and Carter (6). Schlehofer notes that Seroepidemiology of AAV infections in cancer patients which showed that cancer patients exhibited antibodies to AAV types −2, −3 and −5 less frequently than controls, although the data was statistically significant only in the case of cervical carcinoma and AAV-2. The paper concluded that it might be possible to sensitise cells to chemotherapy or irradiation by infecting them with AAV. Again AAV Rep was considered a likely candidate for tumour-suppressive effects. De la Manza et al reported that AAV-2 capsids containing incomplete virions (DI particles) retaining the terminal repeats could supress formation of tumours in hamsters in response to infection with adenovirus-12. Purified AAV DNA injected into animals did not reduce tumour incidence but sheared AAV-2 DNA and DI particle DNA did, particularly that only containing the terminal DNA. The authors here refer to inhibition of adenovirus 12 tumorigenesis.

In contrast to that which has gone before, the present inventors have now determined that nucleic acid containing bases that are unpaired, particularly DNA and particularly that in a relatively stable form such as in AAV terminal DNA, is capable of selectively killing cells that lack effective p53 function, that is the function of p53 that maintains cells in G2 phase. This is significant in so far as it provides a curative therapeutic use of AAV terminal DNA and similar structures containing unpaired bases, and not just a prophylactic use of AAV. Such prophylactic use would require AAV to be adminsitered continuously in order to avoid elimination of active, eg. by integration into the cells genome or nuclease activity. The therapeutic now provided is effective when administered when a tumour has been detected, a facility not at all appreciated by the prior art, potentially for all p53 deficient tumours, or for the purpose of eliminating cells rendered p53 deficient by infection, particuarly but not exclusively by viruses.

The present inventors have now determined that this structure elicits a DNA damage response which in the absence of p53 activity leads to cell death, probably by apopotosis. The inventors investigations indicate that DNA introduced into cells in this way can activate signalling pathways that lead to G2 arrest or cell death, in the absence of damage to cellular DNA. This system presents a novel principle of delivering DNA of unusual or modified structures into cells to selectively eliminate those lacking in p53 activity. However, the inventors have determined that this principle may be applicable to other combinations of tissues and looped/single stranded DNA delivery vehicles, whether these be viral or otherwise.

The experiments of P M Ogston have shown that the presence of p53 activity is clearly required to maintain the G2 block and prevent death of AAV-infected U2-OS and Saos-2 osteoblasts. It was suggested that viral Rep, assciated with AAV-2 DNA could cause this effect.

The inventors have now confirmed that not only the viral capsid proteins, but also viral Rep protein and a combination of these, were unable to elicit such effects. Rather, the similarity between the effects of AAV and those observed when cellular DNA is damaged suggests that the viral DNA, owing to its unusual structure, triggers a DNA damage response. To date the reported p53-associated cellular responses to DNA damage are increases in p21 (14), GADD45 (23) and 14-3-3σ protein levels (18), and inhibition of the cyclin B and cdc2 gene expression (24, 25). The effects induced by AAV have brought to light a new level of regulation in response to DNA damage, and that is the destruction of CDC25C phosphatase. In the absence of this phosphatase, cdc2 remains phosphorylated and therefore inactive. Thus it appears that the underlying feature of G2 arrest induced by DNA damage responses, is the prevention of cyclin B-cdc2 kinase activity.

The notion that p53 can prevent cell death under certain circumstances has gained support from work on cellular responses to DNA damage. How cells that lack p53 activity die when their DNA is damaged remains speculative (26, 27). Because the use of irradiation and genotoxic drugs inevitably causes physical damage to cellular DNA, it was proposed that when these cells attempt to divide, they undergo mitotic catastrophe (18). In the experiments described in the Examples below, the inventors induced DNA damage response using AAV instead of damaging the DNA of the cell, and still observed the death of those cells that lack p53 activity. This is consistent with the idea that cells possess a mechanism triggering cell death if they attempt to undergo mitosis in the presence of a DNA damage response, as happens when cells lack p53 activity.

The present inventors have performed a series of experiments that show that cells that possess p53 activity, when infected with low amounts of AAV, eg 250 moi, arrest briefly at the G2 phase of the cell cycle, after which they re-enter the cycle and resume normal cellular division. On the other hand, cells without p53 activity also arrest at G2 but only for a transient period before undergoing apoptosis. In this series of experiments non-dividing cells were not affected by AAV infection.

Protein extracts from AAV-infected U-20S cells were analysed with the use of antibodies that recognise various proteins that regulate the cell division cycle. In particular the p53 and p21 proteins were found to increase in quantity after AAV infection. The amount of CDC25C protein on the other hand decreased drastically in response to AAV infection while inhibition of proteosome activity prevented the disappearance of the CDC25C protein U2OSp53DD cells which have a deficit of p53 did not contain reduced amounts of CDC25C protein when infected with AAV. The quantity of most other proteins analysed did not fluctuate in response to AAV infection.

When activity of U2OS cell ATM protein, which is normally activated in the event of DNA damage, was inhibited by caffeine, the cells failed to arrest at the G2 phase of the cell cycle when they were infected with AAV. Under normal conditions cyclin B-cdc2 kinase activity increases steadily after completion of DNA synthesis. As a result of AAV infection, the activity of this kinase failed to increase.

In response to AAV infection, the amount and activity of p53 protein was augmented, causing an increase in the amounts of p21 protein. This is likely to be a result of the activation of the ATM kinase. The CDC25C protein, which is a crucial activator of cellular division, is targeted for degradation via the proteasomal pathway. This appeared to occur only when p53 activity is present in the AAV-infected cell. The result of these effects is the inhibition of the cyclin B-cdc2 kinase activity and hence the inhibition of cellular division (G2 arrest). The features of AAV's effects on cells are reminiscent of those induced by DNA damage.

Thus the present invention provides as its focus delivery of a DNA damage signal to a p53 activity deficient cell, such that the cell dies, probably through apoptosis, without the need to damage its native DNA, and advantageously, without risk of damaging DNA of adjacent p53 competant cells.

Viral entry into the cell is required for production of the aforesaid AAV-induced effects. Inactivation of the virus with UV enhances the potency of the virus while the viral capsids alone or the capsids together with Rep proteins were not able to recapitulate the effects of the fill virus on cells. In addition, neither the synthesis of AAV proteins, nor the replication of the AAV DNA was required for the observed effects of AAV on cells. Instead, AAV DNA, which is single-stranded with two hairpin-like structures at both ends, appears to be responsible for inducing a DNA damage response in the cell, similar to that induced by a DNA damaging agent. When cloned double-stranded AAV DNA was introduced into cells by means of transfection, or cells were infected with UV-irradiated adenovirus, a double stranded DNA virus, the cells did not arrest at G2 or die.

Thus in a first aspect of the present invention there is provided a method of killing a cell that is lacking in effective p53 protein activity, particularly as compared to wild type, characterised in that it comprises delivering to the cell a single stranded and/or looped DNA containing at least one unpaired base, the DNA being in a form that is internalised by the cell. Preferably the method selectively kills the cell lacking in p53 protein activity in the presence of a background population of cells having an effective p53 protein activity. Preferably the cells are of mammalian type and more preferably are human. More particularly the cell is a dividing cell and the background population is preferably non-dividing. Thus whereas a cell may be infected by the DNA of the invention when not dividing, that cell will be killed when it divides if p53 is not functional.

Preferably the single stranded DNA is in a form that is resistant to being converted to double stranded DNA in a target cell, ie a cell of the type to be killed, eg a Saos-2 cell. AAV DNA is an example of this, particularly when UV-irradiated to reduce its already restricted replication capability. Preferably, any DNA including a single stranded portion with at least one region of un-basepaired DNA and lacking sites required for binding of any obligatory enzymes or organelles necessary for DNA replication would, by the present invention, suffice.

Preferably the DNA is in the form comprising a length of single stranded DNA in which no base pairing occurs, this being at least of one base long. Single stranded DNA may be in a form which comprises single stranded loops within double stranded DNA, but conveniently all the DNA is single stranded. The DNA might also be in the form of loops that, while double stranded in the sense that complementary bases are paired with each other in a conventional double stranded DNA basepair relationship such as shown in FIG. 1(a) of the figures herewith, these strands have unusual junctions where the adjacent base pairs are not always adjacent in the DNA sequence.

By looped DNA is meant a single strand of DNA that is base paired with itself over all or at least part of its length. Thus part of the single strand may not be base paired to another part of the single strand, but may be base paired with other DNA on a separate strand. The base pairs in the loops in the case AAV DNA are all on the same strand of DNA and comprise hairpin loops, in so far as the loops are ‘tightly formed’ and do not comprise much DNA in unpaired form. It will be apparent to those skilled in the art that such loops may be produced in double stranded DNA where one strand has complementary regions base paired with each other.

While one preferred form of looped DNA will be AAV, particularly AAV-2, DNA, it will be possible to use other DNA sequences that form similar loops, all that is required being internal palindromes that are capable of pairing within the same strand while leaving at least one base, most readily seen to be an internally situated base within the strand, unpaired. In a further example, completely circular DNA, where there is no 3′ or 5′ end, may be used.

Preferred forms of the invention will provide such DNA in a stabilised form with respect to nucleases and other agents that would degrade it. Such modifications will be known to those skilled in the art of oligonucleotide chemistry, particularly by modification of the phosphodiester groups of the DNA backbone, at least at one or both of the 3′ and/or 5′ ends, by replacing them with analogous but more nuclease resistant groups such as peptide, methylene or methylimino groups, but most preferably by phosphorothioate groups. Such technology is provided on contract research basis by companies such as Molecula Research Laboratories, 13884 Park Center Road, Herdon Va. 20171, USA (Molecula is correct spelling) or Metabion, Len-Christ-Strasse 44, D82152 Planegs-Marisried, DE. Many other companies offer custom synthesis of DNA oligos using phosphothioate nucleotides and, while it may be preferred to use all the bases in such from, it will be realised that routine experimentation will allow the best combination of natural and phosphothioate bases in a given poly or oligonucleotide of the invention for the purposes of increasing stability in vivo while not affecting ability to enter cells and maintaining good pharamcokinetic profile. Alternatively the DNA might be rendered resistant to degradation by crosslinking, eg. by UV or chemical crosslinling.

By effective p53 protein activity is particularly meant the ability to bind to DNA or prevent cell division and particularly both. Thus loss of activity may be due to lack of expression of an encoded effective p53 or by mutation of p53 such that one or both activities are lost in the mutant protein. Particularly p53 activity is that which maintains a cell within the G2 phase of the cell cycle.

The single stranded DNA may be in a form that is internalised by all cells, mammalian cells or just human cells, whether lacking (p53−) or having p53 intact (p53+), but more typically will be in a form that is internalised by a sub-population of mammalian or human cells, optionally including both p53− and p53+cells. For example it may be internalised by a sub-population of cells of a particularly tissue type, ie. lung, colon, liver, skin, bladder, CNS, blood (ie. lymphocyte), cervix, neck or bone. Other tissue types that are subject to presence of tumour cells or which are subject to infection that leads to depletion or reduction in p53 activity as compared to non-tumour or non-infected cells will occur to those skilled in the art.

For internalisation purposes the single stranded DNA is conveniently in a form attached to or associated with a moiety that binds with a target cell wall and thus facilitates entry of DNA into the cell, more conveniently being the form of adeno-associated virus, whose protein is capable of using a cell surface receptor for entry into the cell. It will be realised that other proteins from other viruses will also provide ability to enter into cells using different cell surface receptors. Examples of such proteins are capsid or fibre proteins; eg. L1 or L1/L2 protein from Human Papilloma Virus (HPV) assembles into capsids which are internalised by cells and which may be filled with single stranded DNA, eg. of AAV type, preferably AAV single stranded DNA that has been rendered less able to form double stranded DNA by damaging treatment, eg with radiation such as UV. Any other viral capsid protein that is capable of being internalised by cells may also be used to encapsulate the single stranded DNA; examples include adenovirus, herpes virus, HIV, measles, EBV, HCV, MSV-2 etc. Also of use will be viral fibres, such as those of Ad 5, or Ad 40 or 41 (eg. for targeting colon cells) which may be attached to the capsid protein or some other delivery vehicle, eg liposomes, in order to faciltitate internalisation. Such other vehicles may be provided with a moiety that helps internalisation.

It will be realised that it will be desirable to maintain the single stranded form of the DNA within the target cell long enough for the cell to begin apoptosis. In the case of AAV the DNA is protected from degredation by its structure alone, eg. by the end loops. Other such mechanisms are available to those skilled in the art, such as use of DNA mimics, eg. isosteres, and it will be possible to merely conjugate the single stranded DNA with one or more end loops or a degredation resistant mimic to a moiety that is capable of leading to its internalisation in cells.

It is further possible to condense DNA with cationic peptides. The structure of the cationic peptides allows the attachment of ligands for targeting purposes and further peptides to decrease immune responses, eg multiple glycine peptides, eg as available from Cobra Therapeutics. Although the efficiency of this system in vivo can be relatively low, Cobra have developed one system based on the peptide, code name CL22, which is very effective in delivering DNA to a wide variety of cells in vitro.

A further cationic ligand for targeting is a polylysine core, such as that described in Canadian Patent Application 2,251,691 and its US equivalent WO 97/35873, which are incorporated herein by reference. Such core includes a central lysine containing moiety which in turn links to further lysines which in turn are condensed to the oliginucleotide incorporating the un-paired base or bases.

A still further targeting moiety, which can be linked to the DNA or its carrier liposome or capsid, are penetratins such as are described by Derossi et al trends in Cell Biology (vol 8) Feb 1998., p8487, which are capable of being coupled to lipophilic molecules such as DNA and facilitate crossing of the cytoplasmic membrane. Other targeting examples are taught in WO 91/18981. Both these references are incorporated by reference herein.

Although it is believed that such DNA or conjugated DNA as described enough will be effective in many cases, the efficacy of the DNA might be improved by including within it a nuclear localisation signal, such as that of AAV, eg AAV-2, itself. This will enhance passage of the damage response to the cell nucleus.

The present invention thus provides methods of killing p53 activity deficient cells, methods of treating individuals subject to p53 activity deficiency associated disease, use of DNA comprising un-paired single stranded DNA in manufacture of medicaments, such single stranded DNA for use in therapy and compositions comprising such single stranded DNA all as set out in the claims attached and herein above.

Dose of virus or un-paired single stranded DNA to be administered for killing the target p53 deficient cells in vivo, in humans or animals, will depend on the route of administration. For live virus, this may typically be of the order of from 10² to 10¹³, more preferably 10⁴ to 10¹¹, with multiplicities of infection generally in the range 0.001 to 100. Where non-viable virus or non-replicating DNA is used the dose may be equivalently higher, based upon a genomic weight of AAV DNA.

Typical doses of DNA adminstered to patients, even in forms unconjugated to targeting moieties, such as with purified AAV-2 terminal DNA, eg. the terminal 145 bases, will be of the order of 0.01 μg to 100 mg per kilogramrnme, more preferably 0.1 μg to 1 mg per kilogramme, preferably intravenously in a sterile and pyrogen free saline.

The approach of the present invention to targeting cancer cells or cells infected with p53 inhibiting viruses, such as HIV16 or HPV18, has two advantages: (i) only cells that lack p53 activity are killed, and (ii) no damage to cellular DNA is involved. The extension of this principle to other combinations of viruses and cell types as set out above would also provide an additional level of specificity in targeting different tissues.

Currently used methods to induce cell death in cells lacking p53 activity include treatment with DNA damaging agents such as radiation and drugs. The present inventors findings provide an alternative with a number of advantages. Results from their experiments show that a DNA damage signal can be elicited in cells without deliberately damaging the cells' own DNA. This can be achieved by introducing DNA with unusual structures, such as AAV DNA, into cells. Further advantages of this method over existing ones or those being presently developed are listed below:

(i) Viruses are presently the most effective means available to deliver DNA into cells.

(ii) Viruses are also naturally selective in the tissues they infect. This presents the possibility of using a panel of viruses (natural or modified) to target tumours based on their tissue origin, a means not available to present day cancer therapy.

(iii) The problem of multiple-drug-resistance, which limits the effectiveness of chemotherapy, does not apply to this technology.

(iv) The damage to cellular DNA by current cancer therapy can result in the emergence of mutant cells. This will not be a problem with this technology since it is not based on damaging cell DNA.

(v) Since the delivered DNA itself is the causative agent, this technology side-steps difficulties faced by protein-based or gene expression-based approaches to cancer therapy, such as promoter specificity, efficient expression of protein, toxicity of protein to non-tumour cells etc.

(vi) This method is unlikely to pose a safety problem because AAV is not associated with any disease.

(vii) Non-proliferating normal cells in close proximity to a tumour are not endangered since this method does not harm quiescent cells. Dividing cells with p53 activity will either be arrested momentarily before resuming their normal activity or at most be arrested for longer periods without being killed. Hence the possibility of damage to surrounding cells is minimal.

(viii) Since in preferred forms of the invention the viruses are inactivated prior to use, viral transcription, replication and viremia will not occur. Therefore, there would not be the problem of possible homologous recombination with wild-type virus, as may be the case for other viral-based therapy.

The present invention will now be described further by way of illustration only by reference to the following non-limiting figures, sequence listing and examples. Further embodiments falling within the scope of the claims attached hereto will occur to those skilled in the art in the light of these

### FIGURES

FIG. 1: Shows effects of AAV-2 infection on osteosarcoma cells' Schematic representation of AAV DNA (a) Saos-2 (b) or U2OS (e) were infected with AAV at a multiplicity of infection (MOI) of 5000. Condition of cells at 200× magnification 2 days (c and f) or 5 days (d and g) after infection.

FIG. 2: DNA content of cells after AAV infection. Cells were infected with AAV at an MOI of either 250 (a and b) or 5000 (c to n). After the indicated times, cells were harvested, fixed in cold 70% ethanol and stained with propidium iodide. DNA content was measured by fluorescence activated cell sorter by flow cytometry.

FIG. 3: Illustrates apoptosis and protein analysis of AAV-2 infected U2OS and Saos-2 cells

FIG. 3a: Shows FACS analysis of Annexin V in uninfected (left column) and AAV-infected (two days post-infection, right column) Saos-2 cells. The circles area represents apoptotic cells

FIG. 3b: Shows Western blots for U2OS cells infected with retroviruses expressing p53DD, extracts prepared and analysed using antibodies to p53 (DO-1), p53DD (Pb421) and p21.

FIG. 3c: Shows p53 levels in extracts of primary human osteoblasts (NHO) and E6-expressing NHO (NHOE6) analysed using antibodies to p53 (DO-1).

FIG. 3d: Illustrates p53 and p21 protein levels in U2OS at designated tirne points after AAV infection determined using Western Blotting.

FIG. 3e: Illustrates the activaties of cyclin B-cdc2 kinase of U2OS and Saos-2 cells either uninfected or infected by AAV or after Nocodazol treatment deterinined using Histone HI as a substrate.

FIG. 3f: Illustrates cylcin B and cdc2 proteins in U2OS extracts used in (e) above for cyclin B-cdc2 kinase activities determined using Western Blotting.

FIG. 3g: Illustrates CDC25C, CDC25B and actin levels in extracts of U2OS at various times after AAV infection determined using Western Blotting.

FIG. 3h: Shows analysis of CDC25C levels in extracts of U20sp53DD cells at various times after infection by AAV.

FIG. 3i: Illustrates CDC25C protein levels in AAV-infected U2OS in absence or presence of the proteasome inhibitor NaLLN added to the medium 24 hours post-infection and left for 2.5 hours.

FIG. 4: Involvement of p53 in determining cell fate in response to AAV infection. Western blot analyses of Saos-2 cells that were selected with puromycine after infection with retroviruses expressing pRb (a) or p21 (b) Extracts of U2OS infected with retroviruses expressing pS3DD after puromycine selection were analysed by western blotting using antibodies to p53 (DO-1), p53DD (Pb 421) and p21 (c). The p53 protein levels in extracts of primary human osteoblasts (NHO) and E6-expressing NHO were analysed using antibodies to p53 (DO-1) (d). The p53 and p21 protein levels in U2OS at designated time points after infection with AAV were analysed by western blotting with antibodies to the respective proteins (e).

FIG. 5: Biochemical analysis of G2/M checkpoint regulators in response to AAV infection

(a) Cyclin B-cdc2 kinase assay of U2OS and Saos-2 infected with AAV. (b) Western blot of cyclin B and cdc2 proteins of U2OS extracts used in (a). (c) Western blot of cell extracts obtained from U2OS at various time points after infection by AAV with antibodies against human CDC25C, CDC25B and actin. (d) Western blot analysis of CDC25C in extracts prepared from U2OSp53DD at various time points after infection by AAV. (e) 24 hr after AAV infection, NALLN (a proteosome inhibitor) was added to the medium of the infected U2OS for 2.5 hrs. Cells extracts were analysed for CDC25C protein by western blotting. (f) U2OS were either infected with AAV at MOI of 5000 or treated with 2 μg/ml of Etoposide. Cell extracts prepared 24 hours later were analysed with antibodies against p53, p21 and CDC25C on western blots.

FIG. 6: Shows protein analysis of AAV infected colon carcinoma cells and etoposide treated U2OS (a) where a series of related regulatory proteins as indicated in extracts of AAV-infected HCT 116p53+/+ colon carcinoma cells was analysed by Western blotting. (b) Analysis of CDC25C protein levels in HCT116p53−/− cells after AAV infection and (c) U2OS either infected with AAV or treated with 2 μg/ml etoposide. Cell extracts were prepared 24 hours later were electrophoresed and probed with antibodies against p53, p21 and CDC25C.

FIG. 7: Is a graph showing GFP expressing cells plotted versus days post injection with the agents indicated in the legend showing effect of AAV ITRs (terminal 145 bases of AAV-2 DNA only) microinjected into cells.

### SEQUENCE LISTING

The separeately numbered sequence listing attached has sequences as follows:

SEQ ID No 1: The genomic DNA sequence of AAV-2.

SEQ ID No 2: The sequence of AAV-2 ITRs, the double loop structure found at each end of the cosing DNA of SEQ ID No 1.

SEQ ID No 3: The sequence of a first one of the single loops of AAV-2 genomic DNA as found in SEQ ID No 2.

SEQ ID No 4: The sequence of a second one of the single loops of AAV-2 genomic DNA as found in SEQ ID No 2.

SEQ ID No 5: The sequence of a synthetic cyclic DNA according to the invention.

### EXAMPLES

Methods

Cell Culture and Inactivation of p53 Activity in vivo.

U2OS and Saos-2 cells are obtainable from ATCC as HTB-96 and HTB-85 respectively. These cells were cultured in DMEM supplemented with 10% foetal calf serum. NHO was purchased from “Clonetics”. NHO and NHOE6 were cultured in Osteoblast Growth Medium (Clonetics) supplemented with 10% foetal calf serum and ascorbic acid. DNA encoding the p53DD protein was obtained from Dr. M. Oren and subsequently cloned into the retroviral vector, pBabepuro. Candidate retroviruses were prepared by transfecting pBabepurop53DD into phoenix-A cells (from Dr. G Nolan). 3 ml of the medium harvested 48 hours later were used to infect 1.5 million U2OS cells in the presence of 10 μg/ml polybrene. 24 hours after infection, cells were passed and selected with 1.5 μg/ml puromycine. Retroviruses bearing the HPV16 E6 gene were obtained from S. Lathion and used to infect NHO in a similar manner as described for p53DD.

To inhibit the ATM activity, cells were treated with 2 mM caffeine for the indicated times. AnnexinV analysis was performed according to the instructions of the manufacturer (Boehringer Mannheim).

Inactivation of AAV and Infection of Bone Cells

AAV (5000 MOI) was diluted in 0.5 ml of PBS in a small plastic dish and exposed to 2,400 J/m2 of UV irradiation from a “Stratalinker” (Stratagene). The inactivated viruses were further diluted in 2.5 ml of DMEM (10%FCS) before layering them on cells for 3 hours, after which fresh medium was added up to 10 ml.

Flow Cytometry

Cells were trypsinised, washed with PBS and fixed in 70% ethanol After at least 30 minutes they were centrifuged, the ethanol removed and cells resuspended and incubated in 100 μg/ml RNAse in PBS at 37C. After 30 minutes, propidium iodide was added up to 100 μg/ml. DNA content was measured using Florescence activated cell sorter.

Western Blot and Cyclin B-cdc2 Kinase Assay

Cells were washed twice with PBS and scrapped from tissue culture plates using a rubber policeman. After centrifugation in microfuge, cell pellets were resuspended in 2 volumes of Reporter Lysis Buffer (Promega) supplemented with a cocktail of protease inhibitors (Calbiochem). After incubation on ice for 30 minutes with occasional vortexing, the samples were centrifuged at 12,000 rpm in a microfuge for 10 minutes. The supernatants were collected and protein concentrations measured using the Bradford assay (BioRad). 30 μg of proteins per sample were separated on SDS-polyacrylamide gel and transferred to nylon membrane (Hybond) and analysed with antibodies against p53 (R-Iggo), p21, CDC25C, CDC25B, actin, cyclin B and cdc2 (Santa Cruz). The cyclin B-cdc2 kinase assay was performed as described previously (28)

Injection of Cells

Saos-2, U2OS and U2Osp53DD cells were injected with DNAs which were first filtered using a 0.2 μm filter. PCieGFP contained a CMV promoter that controls expression fo Green Fluorescent Protein gene. The AAV hairpin oligoinucleotide was synthesisd (Microsynth) based upon the sequence of AAV-2 inverted terminal repeats (nucleotide positions 1-145). DNAs pCieGFP 400 μg/ml or pCieGFP 200 μg/ml+hairpin DNA 200 μl/ml) were injected into cells using an Eppendorf Micromanipulator. Four hours post-injection, green cells were visible and cells were counted on successive days

### Example 1

### Use of AAV to Kill p53-Osteosarcoma Cells

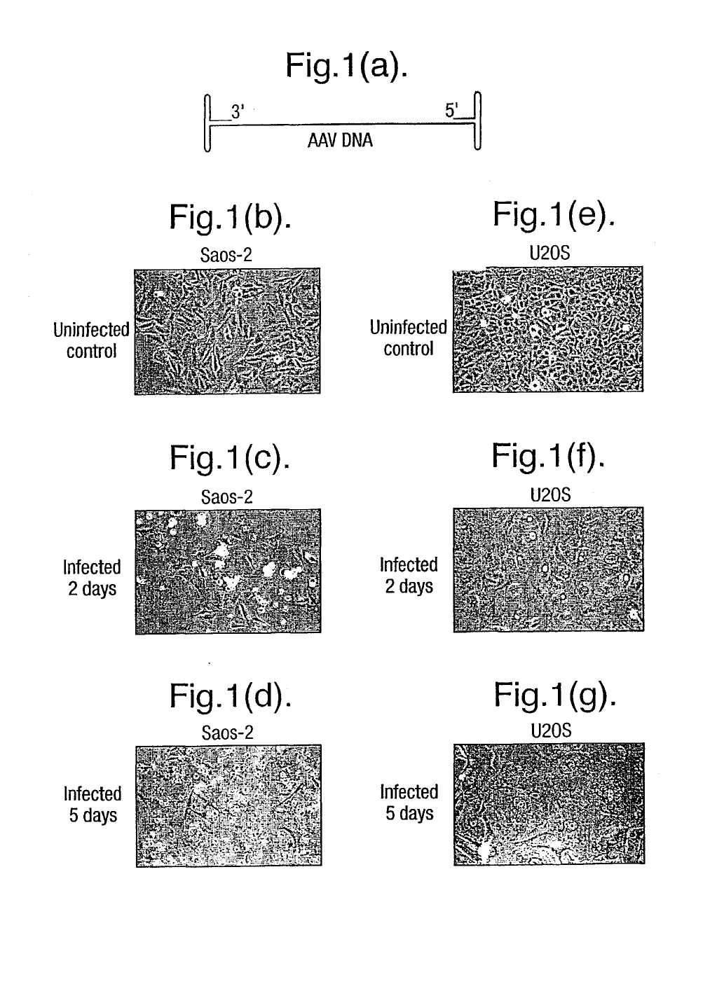

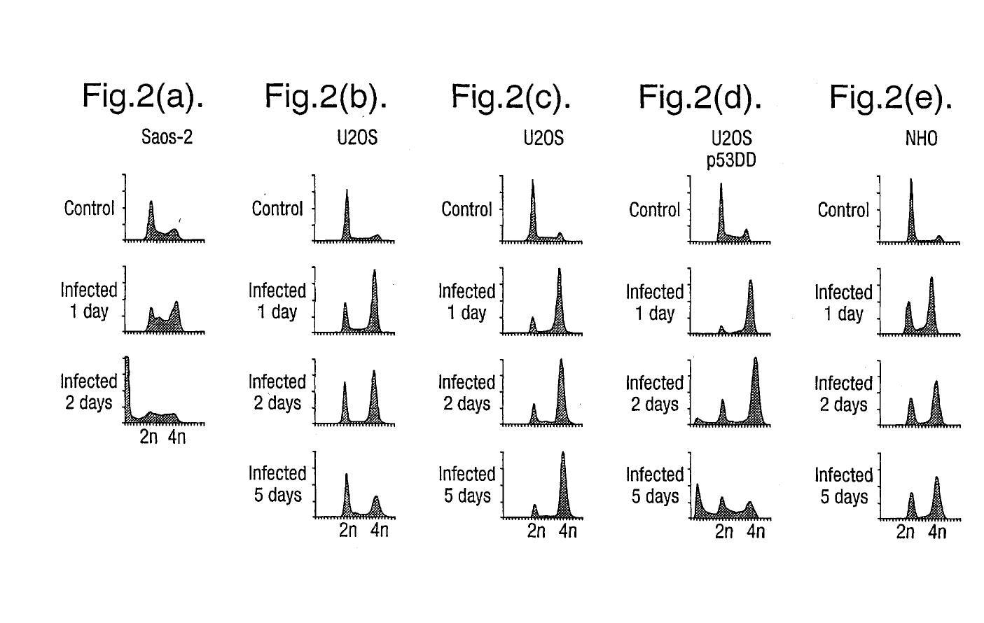

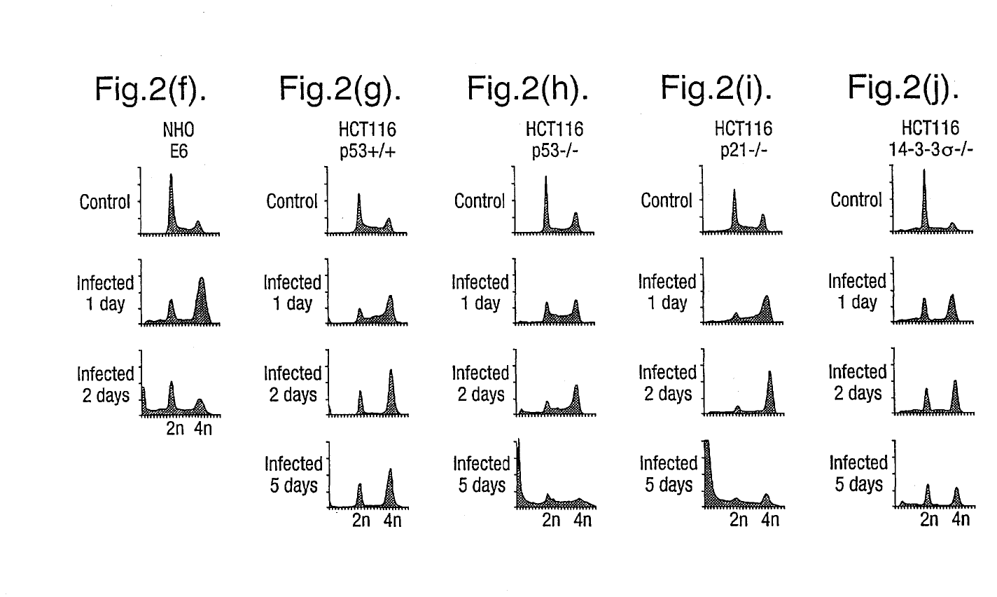

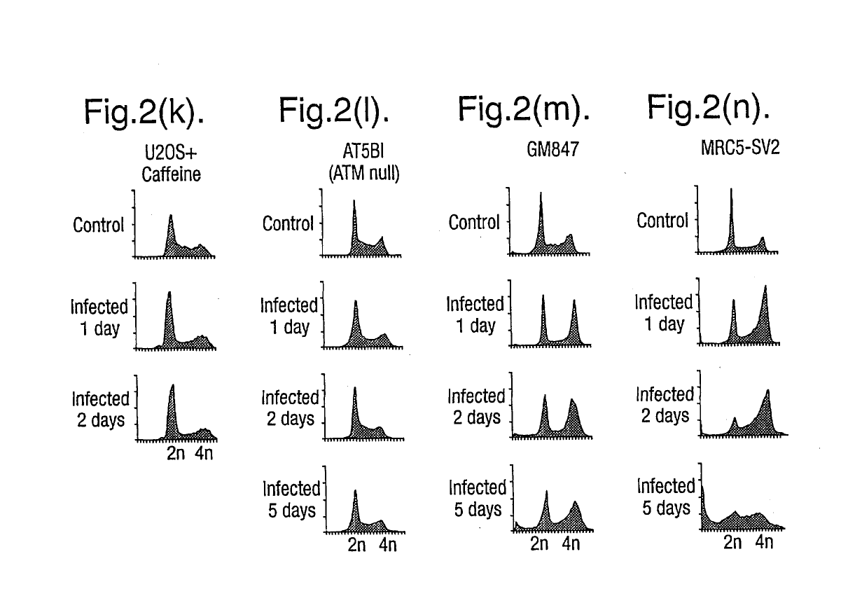

Two osteosarcoma cell lines were infected with AAV-2 in absence of helper virus and were noted to exhibit morphological changes. AAV-infected Saos-2 cells (a p53-null, pRb-null osteosarcoma line) died (FIG. 1b to d), while U2OS cells (which are wild type for p53 and pRb) enlarged to several times the size of uninfected cells (FIG. 1e to g). Measurements of cellular DNA content by flow cytometry revealed that Saos-2 cells, when infected with AAV, accumulated briefly with DNA content greater than 2n. Cell death occurred soon after (FIG. 2a). On the other hand, the majority of U2OS cells arrested with 4n DNA content for several days, after which they re-entered the cell cycle (FIG. 2b). However when higher amounts of AAV were used, most of the U2OS cells arrested in the G2 phase for a prolonged period without subsequent re-entry into the cell cycle (FIG. 2c).

### Example 2

### Use of UV Inactivated AAV DNA to Kill p53-Osteosarcoma Cells

To determine whether AAV replication or the expression of viral proteins were required, we inactivated AAV by ultraviolet (UV) light prior to infection and found that its effect on the cells was not diminished but rather enhanced. We conclude that a component of the virion is responsible. Hence UV-treated AAV was used in subsequent experiments.

### Example 3

### Use of UV Inactivated AAV DNA to Kill U2OS p53+ with p53 Inactivated Using p53DD:

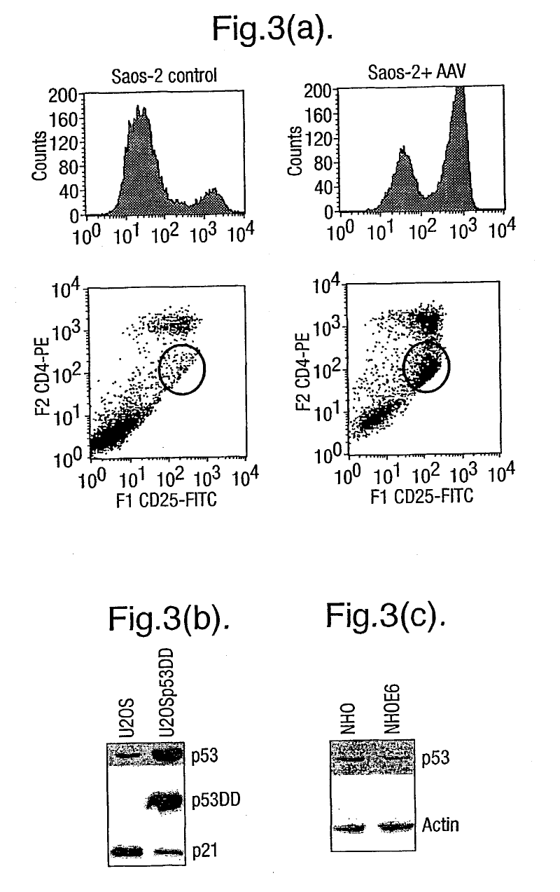

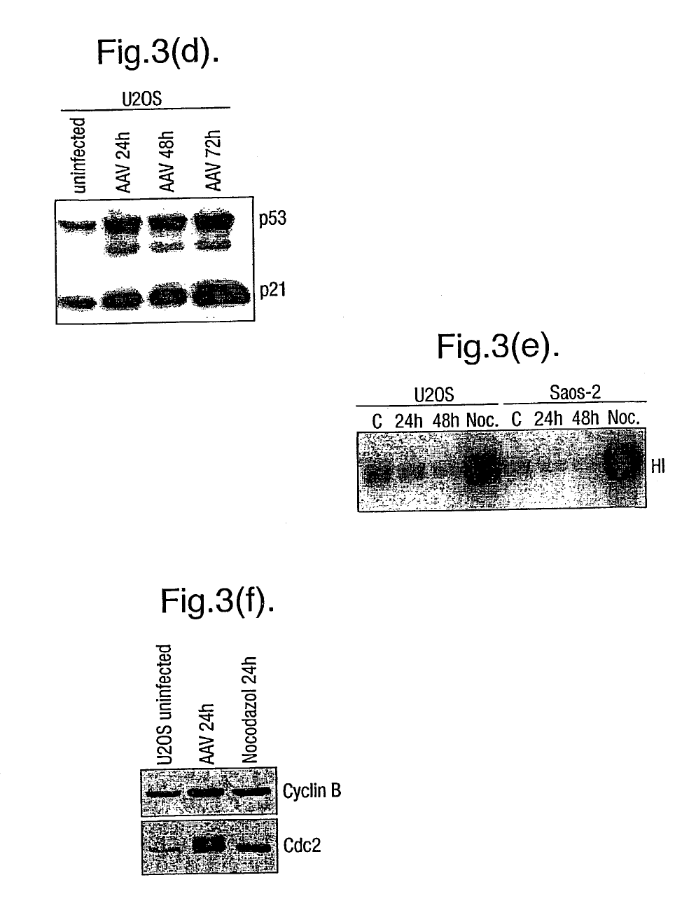

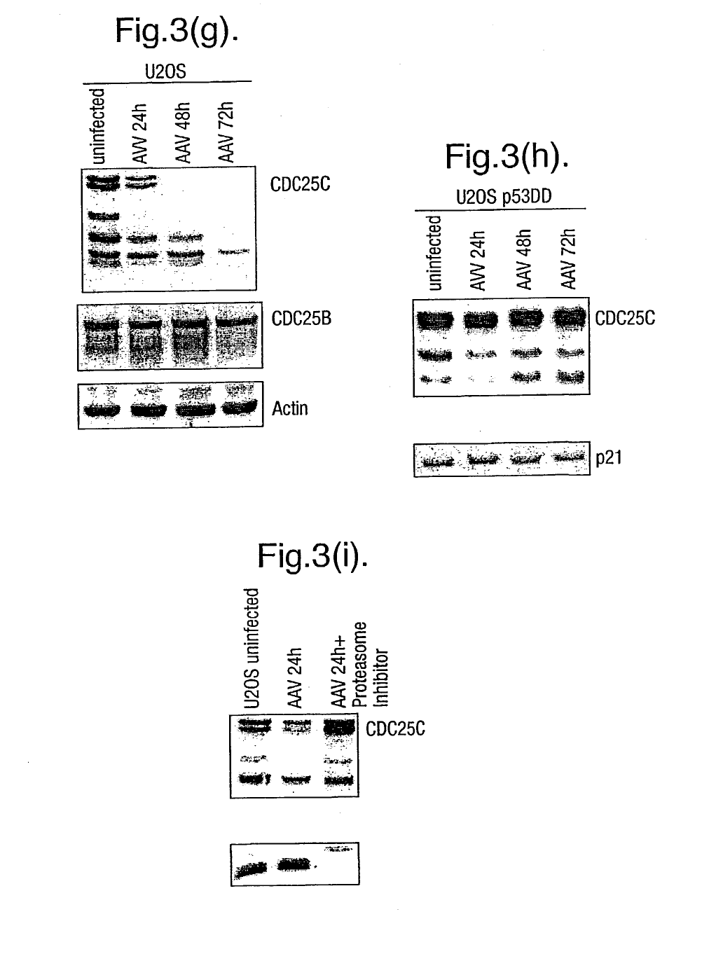

Since Saos-2 cells are null for p53 and pRb, and express very low amounts of p21, we asked whether any of these proteins was responsible for the different reactions (death or G2 arrest) of these two osteosarcoma lines to AAV infection. We expressed p21 or pRb in Saos-2 from retroviral vectors, prior to infecting them with UV-inactivated AAV. The presence of either of these proteins, even at high amounts as determined by western blot analysis (FIG. 3a and b), did not sustain the G2 arrest or prevent Saos-2 from dying (data not shown). To investigate the contribution of p53, we inactivated the p53 protein in U2OS by ectopically over-expressing p53DD, a trans-dominant negative p53 mutant (9). The stabilisation of the endogenous p53 protein and the reduced levels of p21 protein in these cells indicated that the activity of the endogenous p53 was indeed compromised by p53DD (FIG. 3c) (10). Infection of these cells with AAV resulted in a transient G2 arrest followed by cell death (as seen with Saos-2 cells) (FIG. 2d), suggesting that although p53 activity is not necessary to initiate a G2 arrest, it is required to maintain it and prevent cell death.

### Example 4

### Use of UV Inactivated AAV DNA to Kill Normal Human Osteoblasts with p53 Inactivated by IIPV16 E6

To know whether this effect of AAV was unique to Saos-2 and U2OS; or if it was general to bone cells, normal human osteoblasts (NHO) were infected with AAV. These cells also arrested at G2, enlarged and remained so for more than two weeks without dying (FIG. 2e). When the p53 protein in NHO was degraded by expression of the HPV16 E6 protein prior to infection with AAV (FIG. 3d), the cells (NHOE6) arrested at G2 for a short period, before dying (FIG. 2f) Oust as Saos-2 and U2OSp53DD cells did). These observations suggested that the effect of AAV on cell division is not unique to osteosarcomas but is also observable in normal bone cells. In addition, the ablation of p53 activity either by p53DD or HPV16 E6 causes the cells to die when infected with AAV, underlining the importance of p53 activity as the determining factor in the response of dividing osteoblasts to AAV. Consistent with this, western blot analysis showed that the p53 protein in U2OS was stabilised following AAV infection (FIG. 3e). A similar increase was also observed for the p21 protein (FIG. 3e), which is indicative of an increase in p53 activity (10).

### Example 5

### Effect on Cyclin-cdc2 Kinase

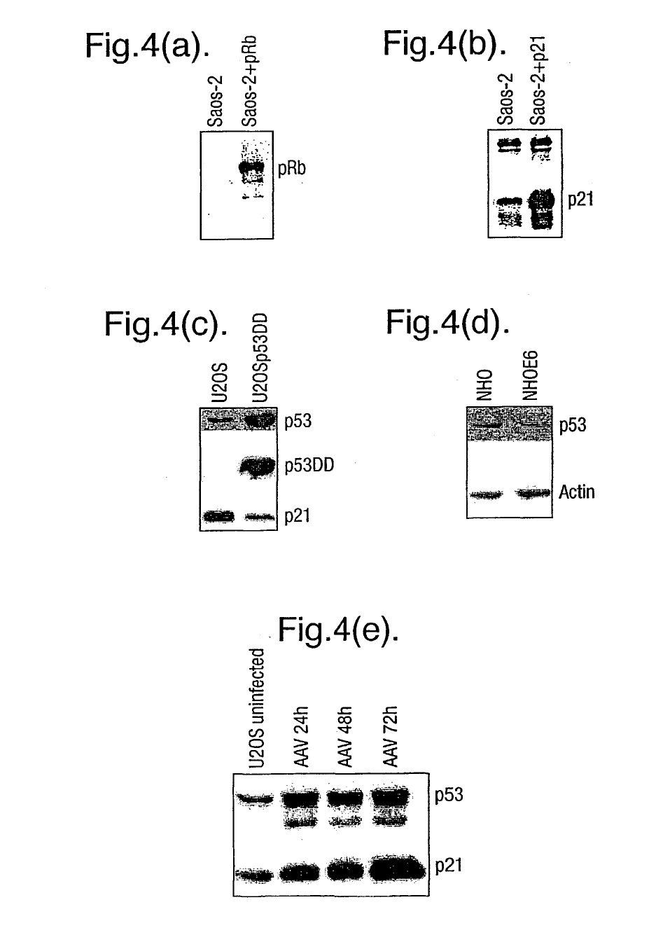

To analyse further the cell cycle block imposed by AAV, activity of the cyclin B-cdc2 kinase was assayed. This enzyme is crucial in triggering the transit of the cell from the G2 phase to mitosis (11). Cells blocked in mitosis by nocodazol exhibit high cyclin B-cdc2 kinase activity (FIG. 4a). However, AAV-infected U2OS and Saos-2 cells, despite having a 4n DNA content possessed cyclin B-cdc2 kinase activity that was even lower than that of the unsynchronised control population, indicating that the AAV-induced block was at the G2 phase (FIG. 4a). Although the activation of p53 and the increase of p21 protein level could contribute to the decreased cyclin B-cdc2 kinase activity, and hence the G2 block (12-14), they are certainly not the only factors responsible because low cyclin B-cdc2 kinase activity was also observed in Saos-2 cells, which lack p53. Since the protein levels of cyclin B and cdc2 in AAV-infected cells were the same as those of mitotic cells (FIG. 4b), the low kinase activity of cdc2 was not a result of lowered protein production. However, following infection of U2OS with AAV, a substantial fraction of the cdc2 protein migrated on gel electrophoresis at a slower rate than the control, indicating that it might be phosphorylated (FIG. 4b). On checking the CDC25C phosphatase, which is crucial for dephosphorylating and activating cdc2 (15), we found that the protein level of this phosphatase decreased dramatically in U2OS in response to AAV infection (FIG. 4c). Interestingly, U2OSp53DD cells, when infected with AAV did not contain reduced levels of CDC25C protein (FIG. 4d). Treatment of AAV-infected U2OS cells with N-acetyl-leu-leu-norleucinal (NaLLN), a proteasome inhibitor (16), prevented the disappearance of the CDC25C protein (FIG. 4e), indicating that the proteasome complex was responsible for the degradation of CDC25C in U2OS. This degradation was specific since the protein level of CDC25B (FIG. 4c) and that of many other proteins tested, were unchanged. We conclude that the destruction of CDC25C protein triggered by AAV is coupled to the presence of functional p53, and is important for the prolonged G2 arrest.

### Example 6

### Comparative Example Control

To determine -which constituent of the AAV particle was responsible for these effects, we infected cells with the individual components of the virus. AAV-like particles were prepared from recombinant baculoviruses expressing VP1, VP2, and VP3. Empty AAV particles, containing the capsid proteins and Rep, but not AAV DNA, were purified from AAV preparations using caesium chloride gradient centrifugation. None of these affected the growth of Saos-2 or U2OS cells. Retroviral-mediated expression of the Rep proteins alone in cells did not change CDC25C protein levels or p53 activity (Saudan et al., submitted). The UV-inactivated AAV used in these experiments is unable to support the synthesis of viral proteins or DNA, indicating that newly synthesised viral proteins were not responsible for inducing these effects. Instead, the results outlined above indicate that the viral DNA is the causative agent. Several lines of evidence suggest that AAV DNA, which is single-stranded with hairpin loops at both ends, can be sensed as abnormal DNA by the cell (17) and trigger a DNA damage response. Firstly, UV-inactivation of the virus prior to use did not reduce but rather increased the magnitude of the effect. By preventing second strand synthesis, UV-treatment preserves the viral DNA in its initial single-stranded form, and thus induces a prolonged activation of the DNA damage checkpoint. Secondly, the cellular response to DNA damage (14, 18) or to AAV infection bears many sirnilarities. In both cases, cells can respond by either establishing a prolonged arrest at G2, if p53 is present, or by pausing briefly at G2 before dying, when p53 is absent

### Example 7

### Comparative Example-Control

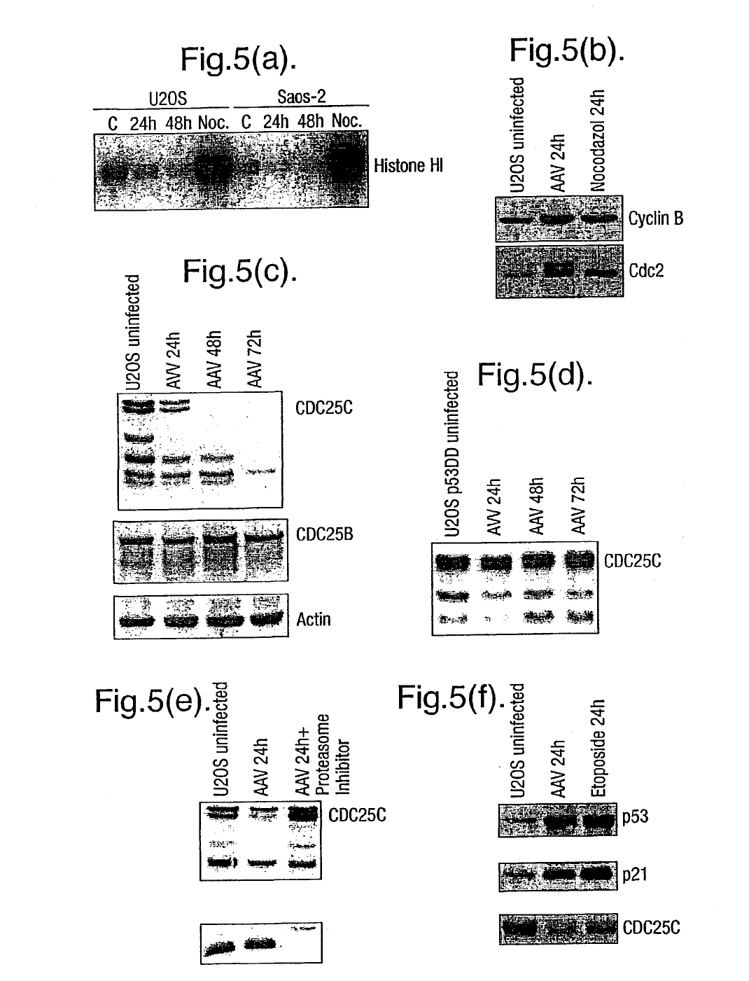

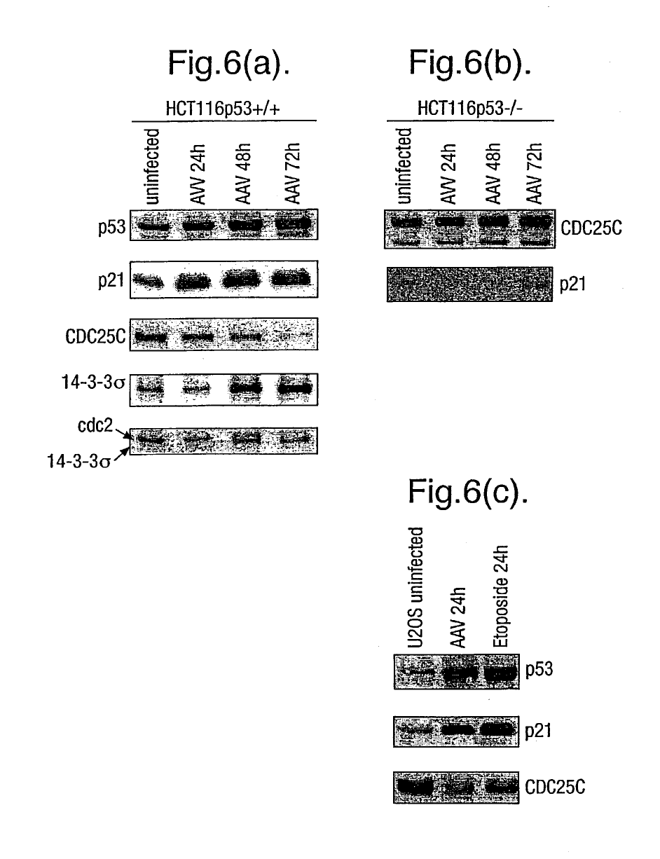

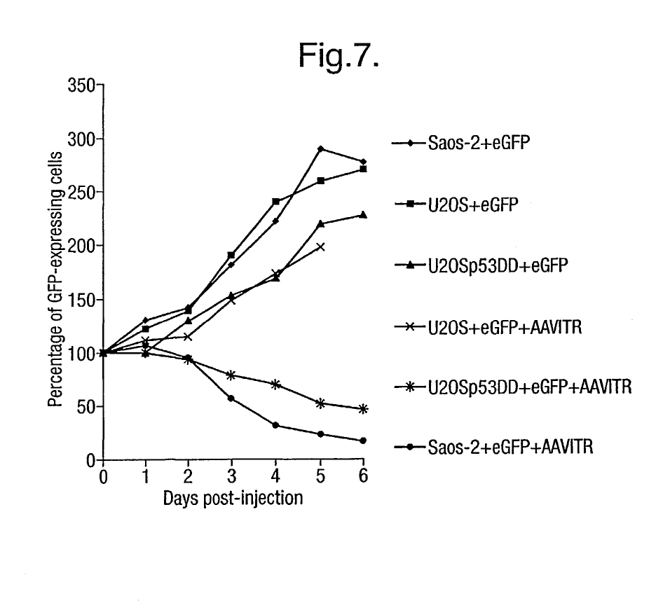

Caesium chloride fractions of AAV preparations were UV-irradiated at 2400J/m² prior to using them infect U2OS cells. After 2 days contents of cellular DNA were measured by FACS analysis. About 60% of cells infected with fraction 3 (see FIG. 5) were arrested in G2. Immunoblots show that Rep proteins were not present in that fraction, but were present in fraction 5 and above, which do not affect cells. Although VP1, VP2 and VP3 were present in fraction 3 they were also present in fractions that produced no response. Fraction 3 contains AAV-DNA.

### Example 8

### Requirement for Internalisation of DNA into Target Cells

When infection with UV irradiated AAV was performed in the presence of heparan sulphate, which blocks the surface receptor by which the AAV enters the cell, the effects of AAV on the cells were diminished in direct proportion to the amount of heparan sulphate present.

When AAV infections were performed in the presence of antibodies against the AAV particle, none of the cells reacted to the virus

When the AAV was inactivated by ultraviolet light, prior to infection, the effect of the virus on the cells were not decreased, but increased.

### Example 9

### Comparison to Etoposide DNA Damage

To confirm that it is the DNA damage pathway that is activated, U2OS cells were treated with etoposide, which is known to damage DNA (19), in place of AAV infection. When the levels of p53, p21 and CDC25C proteins were analysed, they were found to change in a manner identical to that caused by AAV (FIG. 4f), confirming that a DNA damage response was activated. AAV encapsidates either of the complementary viral DNA strands, but in separate viral particles. Isolation of AAV DNA from the particles would not conserve its hairpinned single-strand structure since the complementary strands, once released, can reanneal. Therefore transfection of AAV DNA would not be expected to mimic the effects of AAV infection, a result that was did indeed observed

In the case of AAV-infected osteoblasts, damage is coupled to the destruction of CDC25C protein, but not to an increase of 14-3-3σ protein. In DNA-damaged human colorectal cancer cells the G2 block is coupled to an increase of 14-3-3σ protein levels (18), while in human foreskin fibroblasts, repression of the cyclin B and cdc2 gene expression was reported (24). All these pathways eventually result in the inactivation of the cyclin B-cdc2 kinase activity and the maintenance of the G2 arrest.

### Example 10

### Further Cell Lines

HT1080, human smooth muscle cells were tested and found to be arrested at the G2 phase of the cell cycle when infected with AAV. Human colon carcinoma cell line HCT116 (with wild type p53) were tested and found to arrest at the G2 phase of the cell cycle. When HCT116 p53−/− cells were infected with AAV, they arrested briefly at G2 and subsequently died (see FIGS. 2g and 2h). In the p53+line p53, p21 and 14-3-3σ levels increased while cdc2 and CDC25C decreased (see FIG. 6a). The level in the p53− cells was unchanged as in the p53DD U2OS (see 6b). HCT116 cells lacking p21 failed to sustain G2 arrest and died while those lacking 14-3-3σ sustained arrest with minimal cell death.

FIG. 6c shows that etoposide mimicks this effect. U2OS cells infected in the presence of caffeine, an ATM inhibitor fail to arrest at G2 phase, but continue to proliferate (see FIG. 2k). Consistent with this, ATM null cells (AT5B1, SV40 transformed) were not affected by AAV, while control cells (GM847 and MRC5-SV2) were (see FIG. 2l-n). Thus this is consistent with AAV affecting the cell by inducing ATM-dependent DNA damage response.

Thus AAV is able to induce similar effects in cells of mesenchymal (bone and muscle) and epithelial origin.

### Example 11

### Effect of Hairpin Loop DNA

Saos-2, U2OS and U2OSp53DD cells were microinjected with an oligonucleotide corresponding to the AAV hairpin 145 base sequence (See SEQ ID No 2) with no AAV coding sequence. The Saos-2 and U2OSp53DD cells were killed (see FIG. 7) whereas the U2OS cells survived, illustrating that this un-paired base containing DNA is effective to kill p53-cells. From the earlier work where the purified ITR-DNA was found to suppress tumour formation in repsonse to Ad12 infection in whole Hamsters, it is clear that such DNA may be expected to be internalised by cells after iv injection, without need to microinject individual cells.

### Example 12

### Effect on Tumour Formation

Isogenic HCT116p53−/− and HCT116p53+/+ cell lines were injected under the skin of nude mice followed by injection of AAV or PNBS as control two days later. With the −/− line 100%of the control injections gave rise to tumours, whereas this fell to 17% with AAV treatment. With the +/+ line 80% of the tumours were still formed with AAV, consistent with the findings of de la Maza and Carter ibid.

The effcet of AAV on established tumours was then tested, with size of tumours being 34 to 74% of the controls for −/− lines. With HT29 cells, a further p53-line, AAV caused complete regression of 60% of tumours and reduction in size to 19 to 34% of controls of the remainder.

### Example 13

### Synthetic p53-Selective Cytotoxic DNAs

It will be realised that it is not necessarily the case that one would need to use AAV DNA or loops thereform. The inventors conceive the following proposed DNAs for use of the invention, it being realised that unpaired bases may be substituted for any other bases and paired bases may be substututed by any other basepair, while remaining in the spirit of the invention:

(i) A single stranded DNA having internal palindromic sequence such that all the bases pair up with other bases of the DNA with the exception of a loop end, eg comprising one, two or three unpaired bases, as exemplified by the formula.

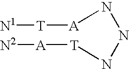

wherein N¹ and N² are hydrogen or equilength oligonucleotide chains basepaired to each other,

the sequences TA and AT linked to the se chains are basepaired to each other in the conventional manner way, and the three bases N at the end are not base paired.

It will be realised that TA and AT may be replaced by CG and GC, GT and TG, TG and GT, GC and CG or AT and TA. or

(ii) Cyclic single stranded oligonucleotides of general formula

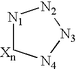

wherein N₁-N₄ and X are independently selected nucleotides and n is an integer from 0 to 10, more preferably 1 to 4, most preferably 1.

One example of such an oligonucleotide is

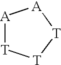

where all the bases are contiguosly linked to each, but one or more or all are not basepaired.

In both cases (i) and (ii) bases may be modified bases that are resistant to nucleases.

Any of the bases, but particularly the 5′ or 3′ bases in the case of (i) may be linked by an ester or amide or other suitable linking bond to a peptide or other targeting moiety if it is desired to change targeting in any way.

Methodology for linking these single stranded oligonucleotides to trageting moieties is that as provided in the following texts, incorporated herein by reference.

Processes for linking DNA to molecules such as biotin and digoxigenin using nirophenyl azido goups and UV radiation are descnied in Forster et al (1985), Habili et al (1987), Agrawal et al (1986), Jablonski et al (1986) and Renz and Kurz (1984), Guesdon (1992), Vialeand Dell'Orto (1992), Reischl et al (1994) and Mansfiled et al (1995)—see Sambrook et al, Molecular Cloning, A laboratory Manual Third Edition, Cold Spring harbour Laboratory Press, Chapter 9 for details of references.

Use of DNA to protein/peptide binding motifs has been employed to assocciate DAN to proteins of peptides, for example use of Gal4 peptide binding motif allows peptides fused to gal4 to be used. (see WO/0026379 and PCT/DE99/03506 incorporated by reference herein.

Signal peptides are coupled to DNA using techniques such as those described in PCT/US95/07539, page 13. Covalent thioester bonding is particularly favoured. DNA can alos be coated and or enmeshed in peptides as described in WO 97/25070, see page 46 incorporated herein by reference.

### UNSPECIFED REFERENCES: INCORPORATED HEREIN BY REFERENCE

1) Srivastava, A., Lusby, E. W., and Bems, K. I. Nucleotide sequence and organisation of the adeno-associated virus 2 genome J. Virol. 45:555-564 (1983)

2) Bems, K. I., and Linden, R. M. The cryptic life cycle of adeno-associated virus. Bioessays 17:237-245 (1995)

3) Kube, D. M., Ponnazhagan, S. and Srivastava, A. Encapsidation of adeno-associated virus type 2 proteins in wild-type and recombinant progeny virions: Rep-mediated growth inhibition of primary cells. J. Virol. 71(10):7361-7371 (1997)

4) Rommelaere, J., and Cornelis, J. J. Antineoplastic activity of parvoviruses. J. Virol. Methods. 33:233-251 (1991)

5) Winocour, E., Callaham, M. F., and Huberman, E. Perturbation of the cell cycle by adeno-associated virus. Virology 167:393-399 (1988)

6) De La Maza, L. M., and Carter, B. J. Inhibition of adenovirus oncogenicity in hamsters by adeno-associated virus DNA. J. Natl. Cancer Inst. 67:1323-1326 (1981)

7) Mayor, H. D., Houlditch, G. S., and Mumford, D. M. Influence of adeno-associated satellite virus on adenovirus-induced tumours in hamsters. Nature 241:44-46 (1973)

8) Hermanns, J., Schulze, A., Jansen-Dürr, P., Kleinschmidt, J. A., Schmidt, R. and zur Hausen, H. Infection of primary cells by adeno-associated virus type 2 results in a modulation of cell cycle-regulating proteins. J. Virol 71(8):6020-6027 (1997)

9) Bowman, T., Symonds, H., Gu, L., Yin, C., Oren, M., and Van Dyke, T. Tissue specific inactivation of p53 tumour suppression in the mouse. Genes and Dev. 10(7):826-835 (1996)

10) Waldman, T., Kinzler, K., W., and Vogelstein, B. p21 is necessary for the p53-mediated GI-arrest in human cancer cells. Cancer Research 55(22):5187-5190 (1995)

11) Nurse, P. Universal control mechanism regulating cell cycle timing in M-phase. Nature 344:503-508

12) Xiong, Y., Hannon, G. J., Zhang, H., Cassa, D., Kobayashi, R., and Beach, D. p21 is a universal inhibitor of cyclin-dependent kinases. Nature 366(6456): 701-704 (1993)

13) Harper, J. W., Elledge, S. J., Keyomarsi, K., Dynlacht, B., Tsai, L. H., Zhang, P., Dobrowolski, S., Bai, C., Connell-Crowley, L., Swindell, E., et al. Inhibition of cyclin-dependent kinases by p21. Mol. Biol. Cell 6(4): 387-400 (1995)

14) Bunz, F., Dutriaux, C., Lengauer, T., Waldman, T., Zhou, S., Brown, J. P., Sedivy, J. M., Kinzler, K. W. and Vogelstein, B. Requirement for p53 and p21 to sustain G2 arrest after DNA damage. Science 282:1497-1501 (1998)

15) Strausfeld, U., Fernandez, A., Capony, J. P., Girard, F., Lautredou, N., Decrancourt, J., Labbe, J. C. and Lanb, N., J. Activation of p34cdc2 protein kinase by microinjection of human CDC25C into mammalian cells. Requirement for prior phosphorylation of cdc25C by p34cdc2 on sites phosphorylated at mitosis J. Biol. Chem. 269(8):5989-6000 (1994)

16) Rock, K. Gramm, C., Rothstein, L., Clark, K., Stein R, Dick, L., Hwang, D., Goldberg, A. L. Inhibitors of the proteosome block the degradation of most cell proteins and the generation of peptides presented on MHC class I molecules. Cell 78:761-771 (1994)

17) Yang, D., van Boom, S. S., Reedijk, J., Farrell, N., and Wang, A. H. A novel DNA structure induced by the anticancer bisplatimun compound crosslinked to a GpC site in DNA Nat. Struct. Biol 2(7):577-578 (1995)

18) Chan, Hermeking, H., Lengauer, C., Kinzler, K. W., Vogelstein, B. 14-3-3σ is required to prevent mitotic catastrophe after DNA damage. Nature 401:616-620 (1999)

19) Wozniak, A. J. and Ross, W. E. DNA damage as a basis for 4′-demethylpipodophyllotoxin-9-(4,6-O-ethylidene-beta-D-glucopyronoside (etoposide) cytotoxicity. Cancer Research 43(1):120-124 (1983)

20) Aguda, B. D. A quantitative analyses of the kinetics of the G2 DNA damage checkpoint system. Proc. Natl. Acad. Sci. U.S.A. 96(20):11352-11357 (1999)

21) Kastan, M. B., Onyekwere, O., Sidransky, D., Vogelstein, B. and Craig, R. Participation of p53 protein in the cellular response to DNA damage Cancer Research 51:6304-6311 (1991)

22) Keubritz, S. J., Plunkett, B., S. Walsh W. V. and Kastan, M. B. Wild type p53 is a cell cycle checkpoint determinant following irradiation. Proc. Natl. Acad. Sci. USA 89(16):7491-7495 (1992)

23) Kastan, M. B. Zhan, Q., el-Deiry, W. S., Carrier, F., Jacks, T., Walsh, W. V., Plunkett, B. S., Vogelstein, B., Fomace, A. J. Jr. A mammalian cell cycle checkpoint pathway utilising p53 and GADD45 is defective in ataxia telangiectasia Cell 71(4):587-597 (1992)

24) Passalaris, T. M., Benanti, J. A., Kiyono, T. and Galloway, D. A. The G2 checkpoint is maintained by redundant pathways. Mol. Cell. Biol 19(9):5872-5881 (1999)

25) Taylor, W. R., DePrimo, S. E., Agarwal, A., Agarwal, M. L. Schonthal, A. H., Katula, K. S. and Stark, G. R Mechanisms of G2 arrest in response to overexpression of p53. Mol. Biol. Cell 10:3607-3622 (1999)

26) Finkel, E. Does cancer therapy trigger cell suicide? Science 286: 2256-2258 (1999) 27) Brown, J. M. and Wouters, B. G. Apoptosis, p53, and tumour cell sensitivity to anticancver agents Cancer Research 59: 1391-1399

28) Fournier, N. Raj, K., Saudan, P., Utzig, S., Sahli, R., Simanis, V. and Beard, P. Expression of human papillomavirus 16 E2 protein in S. pombe delays the initiation of mitosis. Oncogene 8(27):4015-4021 (1999)

1. A method of killing a cell that is lacking in effective p53 protein activity characterised in that it comprises delivering to the cell a single stranded and/or looped DNA including a portion with at least one base that is un-basepaired with another base in a form that is capable of being internalised by the cell with the provisio that the cell is other than a Saos-2 cell.

2. A method as claimed in claim 1 characterised in that the single stranded and/or looped DNA is not configured to expressing a peptide or protein but selectively kills the cell lacking in p53 protein activity in the presence of a background population of cells having an effective p53 protein activity.

3 A method as claimed in any one of the preceding claims characterised in that the cell is a dividing cell.

4. A method as claimed in any one of the preceding claims characterised in that the single stranded and/or looped DNA is in a form attached to or associated with a moiety that binds with a target cell wall.

5. A method as claimed in any one of the preceding claims characterised in that the single stranded and/or looped DNA is in the form of adeno-associated virus or is associated with adeno-associated virus protein.

6. A method as claimed in any one of the preceding claims characterised in that the single stranded and/or looped DNA is in the form of an adeno-associated virus that has been treated such that the DNA is no longer capable of replication or expression in cells.

7. A method as claimed in any one of the preceding claims characterised in that the single stranded and/or looped DNA is in the form of a radiation treated adenovirus asscociated virus.

8. A method as claimed in any one of the preceding claims characterised in that the single stranded and/or looped DNA has a loop of DNA at one or both of its ends.

9. A method as claimed in any one of the preceding claims characterised in that the single stranded and/or looped DNA is associated with a moiety that facilitates internalisation into a target cell.

10. A method as claimed in any one of the preceding claims characterised in that the single stranded and/or looped DNA is encapsulated within a viral protein capsid that is capable of using a cell surface receptor for association with or entry into a target cell.

11. A method as claimed in any one of the preceding claims characterised in that the single stranded and/or looped DNA is associated with, or contained within a vehicle with is associated with, one or more viral fibres which facilitate internalisation of the DNA into a target cell.

12. A method as claimed in any one of the preceding claims characterised in that the single stranded and/or looped DNA is condensed with a cationic peptide.

13. A method as claimed in any one of the preceding claims characterised in that the single stranded and/or looped DNA is associated with or encapsulated within a liposome.

14. A method as claimed in any one of the preceding claim characterised in that the single stranded and/or looped DNA is associated with a penetratin or integrin.

15. A method of treating an individual suffering from a mutant p53 associated cancer, or an infection that inhibits cellular p53, comprising administering to that individual a therapeutically effective amount of a single stranded and/or looped DNA as described in the method of any one of claims 1 to 14.

16. Use of a single stranded and/or looped DNA in a form that is internalisable by a target cell that is lacking in effective p53 protein activity cell for the manufacture of a medicament for treating mutant p53 associated cancer.

17. Use of a single stranded and/or looped DNA in a form that is internalisable by a target cell that is lacking in effective p53 protein activity cell for the manufacture of a medicament for treating infections with viruses that inhibit p53 activity.

18. Single stranded and/or looped DNA including a portion with at least one base, internally located with respect to any 3′ and 5′ ends of the DNA, that is un-basepaired with another base, in a form that is capable of being internalised within a target cell, for use in therapy.

19. Single stranded and/or looped DNA as claimed in claim 18 in a form that is resistant to degradation, for use in therapy.

20. Single stranded and/or looped DNA as claimed in claim 19 characterised in that it has a loop of DNA at one or both of its ends, for use in therapy.

21. Single stranded and/or looped DNA in a form associated with a moiety that is capable of binding to a target cell, the target cell lacking in p53 activity, for use in therapy.

22. Single stranded and/or looped DNA as claimed in any one of claims 18 to 21 in a form that is encapsulated within a viral capsid or a liposome, for use in therapy.

23. Single stranded and/or looped DNA as claimed in any one of claims 18 to 22 in a form that is not capable of self replication in cells, for use in therapy.

24. Single stranded and/or looped DNA as claimed in any one of claims 18 to 23 in a form that does not form double stranded DNA in a cell for use in therapy

25. A pharmaceutical composition comprising a single stranded and/or looped DNA including a portion with at least one base, internally located with respect to any 3′ and 5′ ends of the DNA, that is un-basepaired with another base.

26. A composition as claimed in claim 25 charaterised in that the DNA is associated with a moiety that binds to a target cell lacking p53 activity, said DNA not being in the form of AAV DNA.

27. A pharmaceutical composition comprising AAV DNA that has been rendered incapable of forming double stranded DNA in a target cell by exposure to radiation treatment.

28. A composition as claimed in claim 25 to 27 characterised in that the DNA is provided together with a pharmaceutically acceptable carrier in a pyrogen and/or sterile form.
